(19) United States
(12) Patent Application Publication
(10) Pub. No.: US 2005/0203631 A1
(43) Pub. Date: Sep. 15, 2005

(54) Tibial implant with a broad keel and a through post

(76) Inventors: Michael E. Daniels, Warsaw, IN (US); Robert A. Hodorek, Warsaw, IN (US); John E. Meyers, Columbia City, IN (US); John E. Pendleton, Fort Wayne, IN (US); Rohit K. Somani, Winona Lake, IN (US); Gary J. Zello, Columbia City, IN (US)

Correspondence Address: ZIMMER TECHNOLOGY - ROBERTS, P.O. BOX 1268, ALEDO, TX, 76008, US

(21) Appl. No.: 10/993,661

(22) Filed: Nov. 19, 2004

Related U.S. Application Data
(63) Continuation-in-part of application No. 10/797,663, filed on Mar. 9, 2004.

(51) Int. Cl. A61B 17/58

(52) U.S. Cl. 623/20.32

(57) ABSTRACT

A tibial implant apparatus includes a tibial plate having a medial-lateral width, and includes a generally keel-like base extending from the tibial plate. The base has a medial-lateral span within a range of about 0.6-0.9 times the width of the tibial plate. A tibial implant apparatus includes a tibial plate, a base extending from the tibial plate, and an elongated member. The elongated member includes a first portion and a second portion, and is removably attached to the base. The tibial plate and the base cooperatively define a through-channel. The elongated member is inserted superiorly into the through-channel such that the first portion of the elongated member is retained within the through-channel and the second portion of the elongated member protrudes out of the through-channel and extends inferiorly away from the base. A method for anchoring an implant in a proximal tibia is also provided.

### CROSS-REFERENCE TO RELATED APPLICATION(S)

This is a continuation-in-part of U.S. patent application Ser. No. 10/797,663, filed Mar. 9, 2004, entitled “TIBIAL IMPLANT WITH A THROUGH POST.”

### FIELD OF THE INVENTION

The present invention relates generally to the field of orthopaedics, and, more particularly, to a tibial implant.

### BACKGROUND

Total joint arthroplasty (“joint replacement”) is the surgical replacement of a joint with a prosthesis. A typical knee prosthesis has three main components: a femoral implant, a tibial implant, and a tibio-femoral insert. In general, the femoral implant is designed to replace the distal femoral condyles. The femoral implant is typically made from metal. It typically includes medial and lateral rounded surfaces for emulating the medial and lateral condyles, respectively, with a middle section therebetween for emulating the patella sulcus/trochlear region of the distal femur.

In general, the tibial implant is designed to support and align the tibio-femoral insert. The tibial implant is also typically made from metal. It typically includes a substantially planar tray or plate portion (“tibial plate”) for supporting the insert, and an elongated stem extending away from the tibial plate for anchoring the tibial implant in the intramedullary canal of the proximal tibia.

In general, the tibio-femoral insert is designed to replace the tibial plateau and the meniscus of the knee. It is typically somewhat disk-shaped, and typically includes one or more substantially planar surfaces for bearing on the tibial plate and one or more generally concave surfaces for bearing against the femoral implant. The insert is typically made of a strong, smooth, low-wearing plastic.

In a traditional knee replacement, the surgeon makes a rather lengthy anterior incision spanning over the distal femur, the knee, and the proximal tibia; separates the distal femur and proximal tibia from the surrounding tissues; hyperflexes, distally extends, and/or otherwise distracts the proximal tibia from the distal femur to make room for specialized guides and saws; and uses the guides and saws to prepare these bones for receiving the prosthetics. The surgeon may apply cement to the distal femur and/or to the proximal tibia to help hold the femoral implant and/or tibial implant, respectively, in place. Alternatively, cementless implants may be used. Further, the surgeon drives the femoral implant onto the cut surface of the distal femur and drives the stem of the tibial implant generally longitudinally into the intramedullary canal of the proximal tibia. Finally, the surgeon attaches the tibio-femoral insert to the tibial plate and closes the surgical site.

In contrast to a traditional knee replacement, knee replacement through minimally invasive surgery employs, among other things, smaller incisions, which tend to reduce tissue traumas and accelerate post-operative recoveries. However, because minimally invasive surgery reduces the size of the surgical site, it also generally reduces the amount of space available for inserting, aligning, and securing tibial implants having long, unitary stems.

### SUMMARY OF THE INVENTION

The present invention provides a tibial implant apparatus. The apparatus includes a tibial plate having a medial-lateral width. The apparatus further includes a generally keel-like base extending from the tibial plate. The base has a medial-lateral span that is within a range of about 0.6-0.9 times the width of the tibial plate.

The present invention provides a tibial implant apparatus. The apparatus includes a tibial plate, a base extending from the tibial plate, and an elongated member. The elongated member includes a first portion and a second portion, and the elongated member is removably attached to the base. The tibial plate and the base cooperatively define a through-channel. The elongated member is inserted superiorly into the through-channel such that the first portion of the elongated member is retained within the through-channel and the second portion of the elongated member protrudes out of the through-channel and extends inferiorly away from the base.

The present invention provides an implant apparatus for a proximal tibia. The apparatus includes a tibial plate. The tibial plate includes a superior surface, further includes an inferior surface, and defines a through-hole extending from the superior surface to the inferior surface. The apparatus further includes a first means for anchoring the apparatus in the proximal tibia. The first anchoring means is inserted into the through-hole from the superior surface such that the first anchoring means is partially retained within the through-hole and partially extends inferiorly from the tibial plate.

The present invention provides, for a proximal tibia with dense cancellous bone regions, an implant apparatus including a tibial plate. The tibial plate includes a superior surface, further includes an inferior surface, and defines a through-hole extending from the superior surface to the inferior surface. The apparatus further includes a first means, extending inferiorly from the tibial plate, for anchoring the apparatus in at least one of the dense cancellous bone regions of the proximal tibia.

The present invention provides a method for anchoring a first member of a tibial implant and a second member of a tibial implant in a proximal tibia. The method includes anchoring the first member in the proximal tibia, anchoring the second member in the proximal tibia, and coupling the second member to the first member simultaneously with the step of anchoring the second member in the proximal tibia.

The present invention provides a method for anchoring an implant in a proximal tibia. The method includes placing a first member of the implant in contact with the proximal tibia such that a first opening bounding a through-channel defined by the first member is positioned superior to a second opening bounding the through-channel, and further includes anchoring the first member to the proximal tibia. The anchoring includes inserting a post of the second member into the first opening such that the post extends inferiorly from the second opening.

The present invention provides a method for anchoring an implant in a proximal tibia. The method includes anchoring a tibial plate of the implant to the proximal tibia, positioning a post of the implant superiorly to the tibial plate, and extending the post through the tibial plate into the proximal tibia.

The present invention provides a method for anchoring an implant in a proximal tibia. The method includes anchoring a tibial plate of the implant to the proximal tibia, positioning a single post of the implant superiorly to the tibial plate, extending the single post through the tibial plate into the proximal tibia, and screwing a threaded portion of the single post into the tibial plate. The extending includes anchoring a smooth portion of the single post in the proximal tibia.

The above-noted features and advantages of the present invention, as well as additional features and advantages, will be readily apparent to those skilled in the art upon reference to the following detailed description and the accompanying drawings.

### BRIEF DESCRIPTION OF THE DRAWINGS

FIG. 1 shows a partially exploded perspective view of an exemplary apparatus according to the present invention;

FIG. 2 shows a superior plan view of the exemplary tibial implant of FIG. 1;

FIG. 3 shows an inferior plan view of the exemplary tibial plate and the exemplary base of FIG. 1;

FIG. 4 shows an exploded cross-sectional view of the exemplary tibial implant of FIG. 1 and FIG. 2 (taken along line 4-4 of FIG. 2);

FIG. 5 shows an assembled cross-sectional view of the exemplary tibial implant of FIG. 1 and FIG. 2 (taken along line 4-4 of FIG. 2);

FIG. 6 shows an anterior plan view of the exemplary tibial implant of FIG. 1 and FIG. 2;

FIG. 7 shows an inferior plan view of an alternative tibial implant according to the present invention;

FIG. 8 shows an anterior plan view of the exemplary tibial implant of FIG. 7; and

FIG. 9 shows a cross-sectional view of an exemplary through-channel plug according to the present invention.

### DETAILED DESCRIPTION OF EXEMPLARY EMBODIMENT(S)

Like reference numerals refer to like parts throughout the following description and the accompanying drawings. As used herein, the terms “medial,” “medially,” and the like mean pertaining to the middle, in or toward the middle, and/or nearer to the middle of the body when standing upright. Conversely, the terms “lateral,” “laterally,” and the like are used herein as opposed to medial. For example, the medial side of the knee is the side closest to the other knee and the closest sides of the knees are medially facing, whereas the lateral side of the knee is the outside of the knee and is laterally facing. Further, as used herein the term “superior” means closer to the top of the head and/or farther from the bottom of the feet when standing upright. Conversely, the term “inferior” is used herein as opposed to superior. For example, the heart is superior to the stomach and the superior surface of the tongue rests against the palate, whereas the stomach is inferior to the heart and the palate faces inferiorly toward the tongue. Additionally, as used herein the terms “anterior,” “anteriorly,” and the like mean nearer the front or facing away from the front of the body when standing upright, as opposed to “posterior,” “posteriorly,” and the like, which mean nearer the back or facing away from the back of the body.

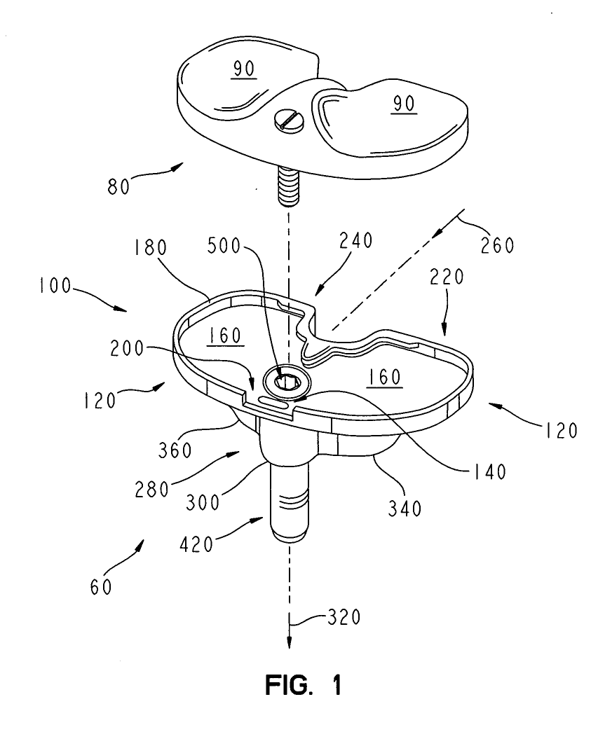

FIG. 1 shows a partially exploded perspective view of an exemplary apparatus 60 according to the present invention. Apparatus 60 includes an exemplary tibio-femoral insert 80. Insert 80 includes a pair of smooth surfaces 90 configured to serve as bearing surfaces against a femoral implant (not shown). In the exemplary embodiment, insert 80 is made from Ultra High Molecular Weight Polyethylene (“UHMWPE”). In alternative embodiments, insert 80 may be made from any other suitably strong, smooth, low-wearing biocompatible material(s).

Apparatus 60 further includes an exemplary tibial implant 100. Among other things, implant 100 is configured to support and align insert 80. Implant 100 includes an exemplary tibial plate 120. Among other things, plate 120 is configured to support insert 80. In the exemplary embodiment, plate 120 is made from a titanium alloy. In alternative embodiments, plate 120 may be made from a cobalt chrome alloy or any other suitable biocompatible material(s). Plate 120 defines a trough or slot 140 and includes a generally planar surface 160 surrounding slot 140. Plate 120 further includes a retaining wall 180 substantially bounding and extending generally perpendicularly away from surface 160. Wall 180 defines an anterior notch 200. It should be appreciated that plate 120 may be thought of as having a medial side or lobe 220 and an opposing lateral side or lobe 240 (relative to an imaginary split line 260). Additionally, it should be appreciated that slot 140, surface 160, wall 180 and notch 200 may facilitate alignment and/or retention of insert 80.

Implant 100 also includes an exemplary base 280 extending from plate 120. Among other things, base 280 is configured to anchor into a proximal tibia (not shown). In the exemplary embodiment, base 280 is made from a titanium alloy. In alternative embodiments, base 280 may be made from a cobalt chrome alloy or any other suitable biocompatible material(s). Base 280 is a generally keel-like structure extending generally inferiorly from and generally medially-laterally relative to plate 120. Base 280 includes a collar portion 300 extending generally inferiorly from plate 120 along an axis 320, further includes a medial generally keel-like portion 340 extending generally inferiorly from plate 120 and radiating generally medially from collar 300, and further includes a lateral generally keel-like portion 360 extending generally inferiorly from plate 120 and radiating generally laterally from collar 300. Base 280 is discussed further below.

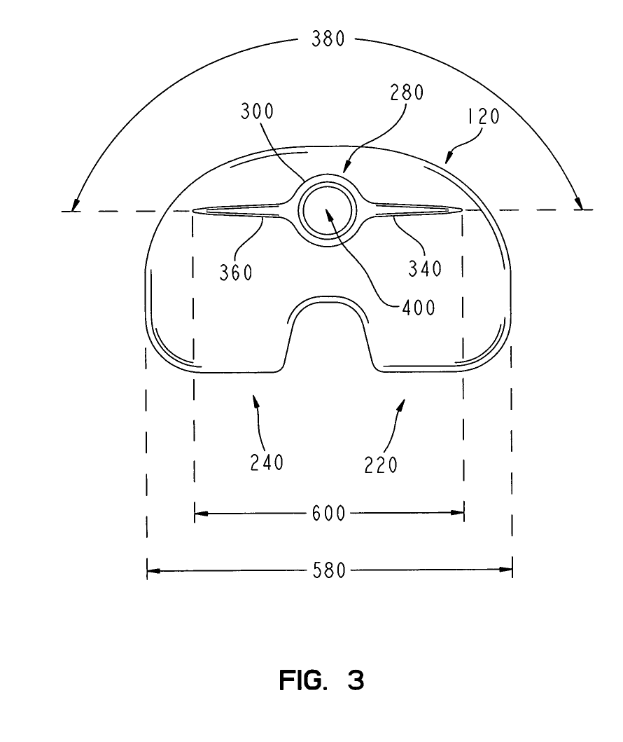

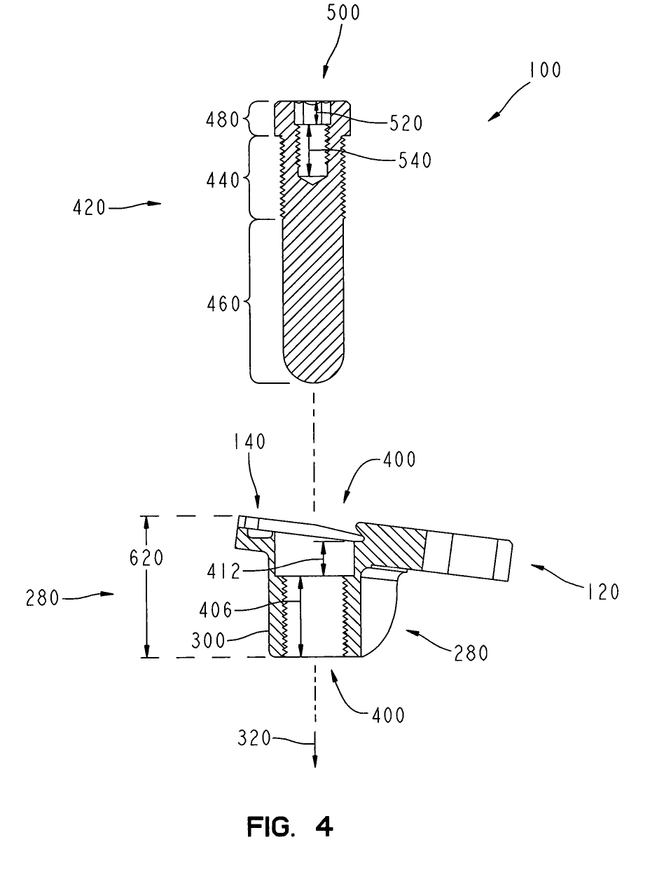

Plate 120 and collar 300 define a passageway or through-channel 400 (see FIG. 3 and FIG. 4) axially extending all the way through surface 160 and collar 300 along axis 320. Through-channel 400 includes a generally cylindrically-shaped screw-threaded portion 406 and a generally cylindrically-shaped non-threaded portion 412 (see FIG. 4).

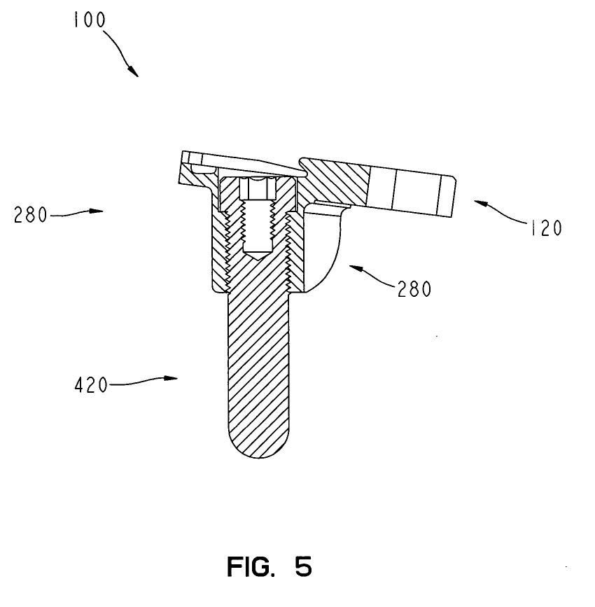

Implant 100 further includes an exemplary extension member or post 420 inserted through and extending from through-channel 400 along axis 320 (see FIG. 4 and FIG. 5). Among other things, post 420 is configured to couple to base 280 within through-channel 400 and, in some embodiments, to anchor into the proximal tibia such that post 420 (and, thus, implant 100) is linearly fixed relative to the proximal tibia along axis 320. In the exemplary embodiment, post 420 is made from a cobalt chrome alloy. In alternative embodiments, post 420 may be made from a titanium alloy or any other suitable biocompatible material(s). Post 420 includes a generally cylindrically-shaped screw-threaded shaft portion 440 screw-coupled to base 280 within portion 406 of through-channel 400 (see FIG. 4). Post 420 further includes a generally cylindrically-shaped non-threaded shaft portion 460 extending generally inferiorly out of through-channel 400 along axis 320, and a head portion 480 generally opposite portion 460 (see FIG. 4). Portion 480 is retained in portion 412 of through-channel 400 (see FIG. 4). In alternative embodiments, portion 440 of post 420, portion 480 of post 420, portion 406 of through-channel 400, and/or portion 412 of through-channel 400 may be alternatively configured to provide a taper coupling or any other suitable coupling between post 420 and base 280. In the exemplary embodiment, portion 460 is about 35 millimeters long. In alternative embodiments, portion 460 may instead be about 65 millimeters long (when greater extension of post 420 into the proximal tibia is desired), may be omitted altogether (when no extension of post 420 into the proximal tibia is desired), or portion 460 may be any other suitable length.

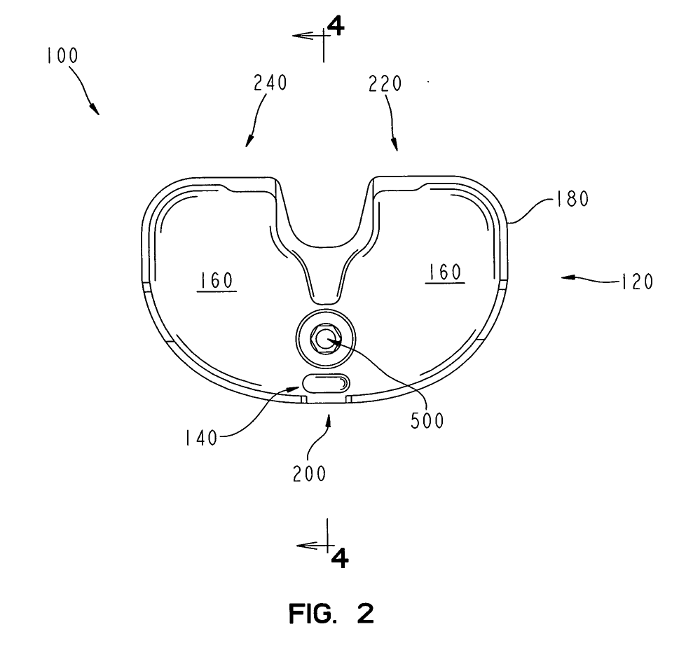

Further, post 420 defines a socket 500 (see also FIG. 2, FIG. 4, and FIG. 5) having a non-circular generally cylindrically-shaped portion 520 opening from head 480 and extending into post 420, and further having a generally cylindrically-shaped screw-threaded portion 540 extending from portion 520 even further into post 420 (see FIG. 4 and FIG. 5). Among other things, portion 520 of socket 500 is configured to facilitate installation of post 420 in through-channel 400 of base 280. Portion 540 of socket 500 facilitates the alignment and/or retention of insert 80. In alternative embodiments, portion 520 may be hexagonally-shaped, torqx-shaped, or otherwise suitably alternatively shaped, and/or portion 540 may be configured for a taper or any other suitable coupling.

FIG. 2 shows a superior plan view of exemplary tibial implant 100. Slot 140, surface 160, wall 180, notch 200, and socket 500, among other things, are all at least partially discernable in FIG. 2.

FIG. 3 shows an inferior plan view of exemplary tibial plate 120 and exemplary base 280. As discernable in FIG. 3, plate 120 has a medial-lateral width 580, and the medial extension of portion 340 of base 280 is disposed from the lateral extension of portion 360 of base 280 by an angle 380 of about 180 degrees. In alternative embodiments, the keel-like portions of base 280 may suitably radiate or fan out from collar 300 at any other suitable angle or angles. In any event, base 280 has a medial-lateral span 600 (from the utmost medial extension of portion 340 to the utmost lateral extension of portion 360) that is within a range of about 0.6-0.9 times width 580 of plate 120. Consequently, span 600 is considerably broader than comparable conventional tibial plate structures such that when anchored in a proximal tibia portion 340 and portion 360 extend into respective dense cancellous bone regions (in line with the natural condylar loads) for enhanced fixation and stability; yet span 600 is also notably smaller than width 580 so as to avoid cortical impingement. Studies have shown that the medial and lateral cancellous bone regions of the proximal tibia are three to five times more dense than the central region surrounding the medullary canal (where many conventional tibial plates are anchored). Collar 300 and through-channel 400, among other things, are also both at least partially discernable in FIG. 3.

FIG. 4 shows an exploded cross-sectional view of exemplary tibial implant 100 (taken along line 4-4 of FIG. 2). As discernable in FIG. 4, plate 120 and base 280 have a combined superior-inferior height 620. In the exemplary embodiment, height 620 is preferably less than 25 millimeters to facilitate installation of plate 120 and base 280 within the tight spacing presented by minimally invasive surgeries. Portion 406 of through-channel 400, portion 412 of through-channel 400, portion 440 of post 420, portion 460 of post 420, portion 480 of post 420, portion 520 of socket 500, and portion 540 of socket 500, among other things, are all at least partially discernable in FIG. 4.

FIG. 5 shows an assembled cross-sectional view of exemplary tibial implant 100 (taken along line 4-4 of FIG. 2). Plate 120, base 280, and post 420, among other things, are all at least partially discernable in FIG. 5.

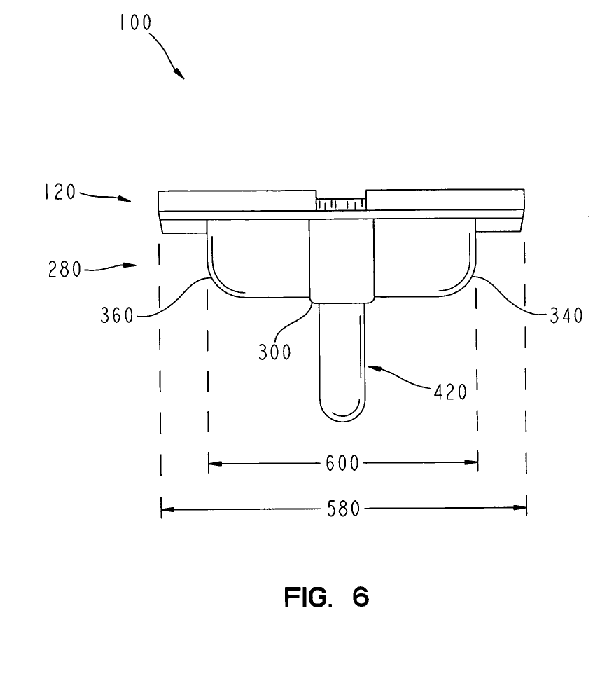

FIG. 6 shows an anterior plan view of exemplary tibial implant 100. Plate 120, base 280 (including portion 300, portion 340, and portion 360), post 420, width 580, and span 600, among other things, are all at least partially discernable in FIG. 6.

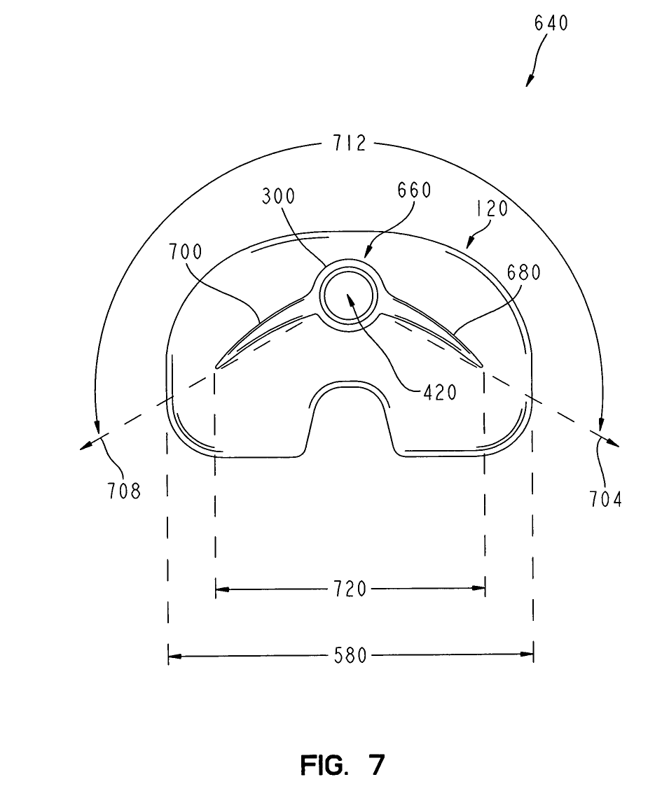

FIG. 7 shows an inferior plan view of an alternative tibial implant 640 according to the present invention. Implant 640 is configured and used identically to implant 100 with the exception that implant 640 includes an alternative base 660 in place of base 280. Base 660 is configured and used identically to base 280 with the exception that base 660 includes an alternative medial generally keel-like portion 680 in place of portion 340, and further includes an alternative lateral generally keel-like portion 700 in place of portion 360. In implant 640, portion 680 extends generally inferiorly from plate 120 and radiates generally medially, generally posteriorly, and somewhat arcuately from collar 300 (in the general direction of line 704), while portion 700 extends generally inferiorly from plate 120 and radiates generally laterally, generally posteriorly, and somewhat arcuately from collar 300 (in the general direction of line 708). The general radiation of portion 680 is disposed from the general radiation of portion 700 by an angle 712. The inventors presently believe that optimal values for angle 712 are within a range of about 210-240 degrees. In the exemplary embodiment, angle 712 is about 225 degrees. Nevertheless, it is noted that in alternative embodiments the generally keel-like portions of base 660 may suitably radiate or fan out from collar 300 at any other suitable angle or angles. Similar to base 280, base 660 has a medial-lateral span 720 (from the utmost medial extension of portion 680 to the utmost lateral extension of portion 700) that is within a range of about 0.6-0.9 times width 580 of plate 120. Consequently, span 720 is considerably broader than comparable conventional tibial plate structures such that when anchored in a proximal tibia portion 680 and portion 700 extend into respective medial and lateral dense cancellous bone regions (in line with the natural condylar loads) for enhanced fixation and stability; yet span 720 is also notably smaller than width 580 so as to avoid cortical impingement. However, it is noted that the arcuateness of portion 680 and the arcuateness of portion 700 provide increased anterior surface area 740 and increased anterior surface area 760, respectively (and correspondingly increased posterior surface areas), for anchoring these portions in the proximal tibia as compared to portion 340 and portion 360. In alternative embodiments, base 660 may include any suitable number of keel-like portions (including none at all in some embodiments) and/or any suitable number of other suitably positioned suitable protuberance(s) (e.g., spikes, vanes, etc.) for suitably anchoring base 660 into the proximal tibia. Post 420, among other things, is also at least partially discernable in FIG. 7.

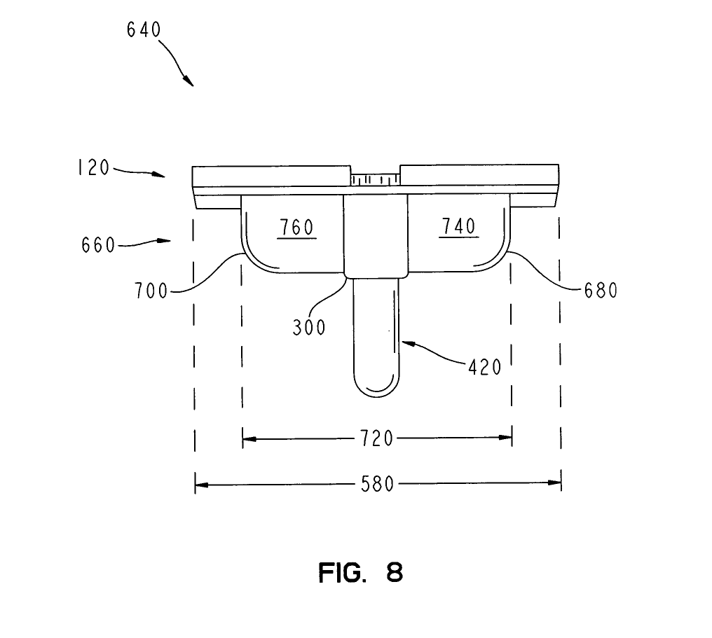

FIG. 8 shows an anterior plan view of exemplary tibial implant 640. Plate 120, base 660 (including portion 300, portion 680, and portion 700), post 420, width 580, and span 720, among other things, are all at least partially discernable in FIG. 8.

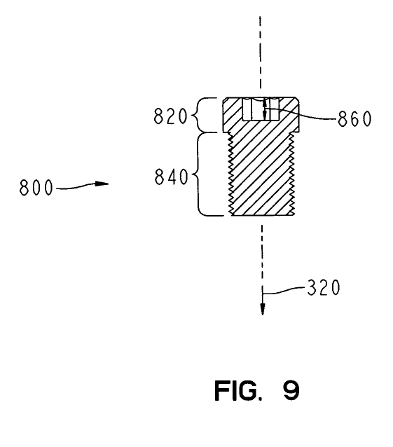

FIG. 9 shows a cross-sectional view of an exemplary through-channel plug 800 according to the present invention. Plug 800 is configured to, among other things, prevent bone cement from seeping into and/or through through-channel 400 during implantation of various embodiments of the present invention. In the exemplary embodiment, plug 800 is made from UHMWPE. In alternative embodiments, plug 800 may be made from any other suitable biocompatible material(s). Plug 800 includes a generally cylindrically-shaped head 820 and a generally cylindrically-shaped screw-threaded shaft 840 extending from head 820. Head 820 defines a non-circular generally cylindrically-shaped socket 860 opening from and extending into head 820. Among other things, socket 860 is configured to facilitate installation of plug 800 in through-channel 400 of base 280. In alternative embodiments, socket 860 may be hexagonally-shaped, torqx-shaped, or otherwise suitably alternatively shaped.

To implant plate 120 and base 280 (or base 660, etc.) in a proximal tibia for a right knee replacement, post 420 is first omitted from through-channel 400. Plug 800 is inserted into through-channel 400 along axis 320 until shaft 840 of plug 800 reaches screw-threaded portion 406 of through-channel 400. A suitable hexagonal drill bit, suitable hexagonal screwdriver head, suitable Allen wrench, or any other suitable torquing tool is inserted into socket 860 of plug 800. The torquing tool is torqued to screw shaft 840 of plug 800 into portion 406 of through-channel 400 until head 820 of plug 800 suitably seats within portion 412 of through-channel 400, and then the torquing tool is withdrawn from plug 800.

Next, the knee joint is opened, the proximal tibia and the distal femur are suitably resected, and the proximal tibia is suitably broached (via suitable minimally invasive surgical techniques or any other suitable procedures) to prepare the proximal tibia to receive plate 120 and base 280. In the present example, such resections and/or other preparations preferably provide on the order of 20 millimeters working or clearance space between the proximal tibia and the distal femur prior to their distraction. The proximal tibia is then distracted from the distal femur as necessary to provide clearance for inserting plate 120 and base 280 into the joint space and for aligning them superior to the proximal tibia. In the present example, this distraction preferably extends the clearance space between the distal femur and the proximal tibia by on the order of 5 millimeters. It should be appreciated that the necessary clearance space may be less than that which would be required to insert and align plate 120 and base 280 with post 420 installed.

If for any reason an application of post 420 is desired (such as, for example, to further anchor base 280 into the proximal tibia), a suitable intramedullary bore is drilled into the proximal tibia (in line with axis 320). For a cemented application, the diameter of the intramedullary bore is preferably large enough to hold a suitable amount of bone cement around portion 460 of post 420 in addition to portion 460 itself. It should be appreciated that for a cementless application, the diameter of the intramedullary bore may be slightly smaller so as to fit suitably snugly around portion 460. However, if post 420 is not desired, then the intramedullary bore is not made.

Next, for a cemented application bone cement is suitably injected into the intramedullary bore (if applicable) and suitably applied to the superior surface of the prepared proximal tibia. Alternatively, the bone cement is omitted for a cementless application. In either event, plate 120 and base 280 are suitably aligned superior to the proximal tibia (with, among other things, through-channel 400 suitably generally coaxially aligned with the longitude of the tibial intramedullary canal along axis 320), and base 280 is suitably forced generally distally into the proximal tibia. Among other things, this anchors base 280 into the proximal tibia. Meanwhile, plug 800 prevents the bone cement (if applied) from seeping into and/or through through-channel 400 (and thus, prevents bone cement from clogging the screw threads within portion 406 of through-channel 400 and/or from flowing superiorly through through-channel 400 onto surface 160 of plate 120.

If post 420 is desired, plug 800 is removed (via reverse application of the torquing tool), post 420 is inserted through through-channel 400 along axis 320 and slid generally distally and generally longitudinally into the intramedullary bore (with finger force) until screw-threaded portion 440 of post 420 reaches screw-threaded portion 406 of through-channel 400. Further, the torquing tool is inserted into portion 520 of socket 500 (of post 420) and torqued to screw portion 440 of post 420 into portion 406 of through-channel 400 (thus forcing post 420 even further generally distally and generally longitudinally into the intramedullary canal) until head portion 480 of post 420 suitably seats within portion 412 of through-channel 400. After securing portion 440 of post 420 in portion 406 of through-channel 400, the torquing tool is withdrawn from socket 500 and base 280 is suitably impacted generally distally into the proximal tibia for a final alignment and anchoring of base 280 and post 420. It should be appreciated that if post 420 is not desired, then plug 800 is simply left in place.

Insert 80 is aligned and/or retained on surface 160 of plate 120 via slot 140 of plate 120, wall 180 of plate 120, notch 200 of plate 120, and/or portion 540 of socket 500 of post 420 (if applicable).

It should be appreciated that the present invention is well suited for use in minimally invasive quadriceps-sparing total knee arthroplasty procedures. In the exemplary embodiment, height 620 optimizes insertion during such procedures (which generally seek to minimize tendon and muscle cutting and generally offer smaller soft tissue windows for inserting implants). The inventors have determined that in most such cases height 620 is, optimally, between 15-25 millimeters. Nevertheless, it should also be appreciated that the invention can also be used in traditional open surgical procedures as well. Additionally, it is noted that span 600 (or span 720) is considerably broader than comparable conventional tibial plate structures such that when anchored in a proximal tibia portion 340 (or portion 680) and portion 360 (or portion 700) extend into respective dense cancellous bone regions (in line with the natural condylar loads) for enhanced fixation and stability (for example, resistance to bending moments, rotation resistance, and lift off resistance); yet, span 600 (or span 720) is also notably smaller than width 580 so as to avoid cortical impingement. Furthermore, it is noted that the potential for distal intramedullary cortical impingement is reduced by the low profile of base 280. It should also be appreciated that the low profile drop-down design of the exemplary embodiment better accommodates bowed tibial intramedullary canals. It is also noted that the locations of portion 340 (or portion 680) and portion 360 (or portion 700) strengthen plate 120 directly under the condylar load bearing areas. Also, it should be appreciated that post 420 provides additional intramedullary stability. In alternative embodiments, longer posts may be employed to provide additional stability when needed/required.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. A tibial implant apparatus, comprising:
a tibial plate having a medial-lateral width; and
a generally keel-like base extending from the tibial plate;
wherein the base has a medial-lateral span being within a range of about 0.6-0.9 times the width of the tibial plate.

2. The apparatus of claim 1, wherein the tibial plate and the base have a combined inferior-superior height being less than 25 millimeters.

3. The apparatus of claim 1, wherein the tibial plate and the base cooperatively define a through-channel, the base includes a first generally keel-like portion extending away from the through-channel, and the base further includes a second generally keel-like portion extending away from the through-channel.

4. The apparatus of claim 3, wherein the first generally keel-like portion arcuately extends away from the through-channel, and the second generally keel-like portion arcuately extends away from the through-channel.

5. The apparatus of claim 3, wherein the first generally keel-like portion extends generally radially outwardly away from the through-channel, and the second generally keel-like portion extends generally radially outwardly away from the through-channel at an angle within a range of about 210-240 degrees from the generally radially outward extension of the first generally keel-like portion.

6. The apparatus of claim 3, further comprising:
an elongated member including a first portion and a second portion, the elongated member being removably attached to the base;
wherein the elongated member is inserted superiorly into the through-channel, such that the first portion is retained within the through-channel and the second portion of the elongated member protrudes out of the through-channel and extends inferiorly away from the base.

7. The apparatus of claim 6, further comprising:
a tibio-femoral insert coupled to the tibial plate.

8. A tibial implant apparatus, comprising:
a tibial plate;
a base extending from the tibial plate; and
an elongated member including a first portion and a second portion, the elongated member being removably attached to the base;
wherein the tibial plate and the base cooperatively define a through-channel, the elongated member is inserted superiorly into the through-channel, such that the first portion of the elongated member is retained within the through-channel and the second portion of the elongated member protrudes out of the through-channel and extends inferiorly away from the base.

9. The apparatus of claim 8, further comprising:
a tibio-femoral insert coupled to the tibial plate.

10. The apparatus of claim 8, wherein the base includes a first generally keel-like portion.

11. The apparatus of claim 10, wherein the first generally keel-like portion extends generally radially outwardly relative to the through-channel.

12. The apparatus of claim 11, wherein the base includes a second generally keel-like portion extending generally radially outwardly relative to the through channel at an angle within a range of about 210-240 degrees from the generally radially outward extension of the first generally keel-like portion.

13. The apparatus of claim 11, wherein the first generally keel-like portion arcuately extends away from the through-channel, and the base further includes a second generally keel-like portion arcuately extending away from the through-channel.

14. The apparatus of claim 8, wherein the first portion of the elongated member is retained in the through-channel at least in part by at least one of screwing the first portion of the elongated member into the base and tapering the first portion of the elongated member into the base.

15. The apparatus of claim 8, wherein the elongated member defines a socket.

16. The apparatus of claim 15, wherein the socket includes at least one of a generally hexagonally-shaped portion and a generally torqx-shaped portion.

17. The apparatus of claim 15, wherein the socket includes at least one of a screw-threaded portion and a tapered portion.

18. The apparatus of claim 8, wherein the base includes a plurality of protuberances positioned generally radially outwardly away from the through-channel.

19. The apparatus of claim 18, wherein the first portion of the elongated member is retained in the through-channel at least in part by at least one of screwing the first portion of the elongated member into the base and tapering the first portion of the elongated member into the base.

20. The apparatus of claim 18, wherein the elongated member defines a socket.

21. The apparatus of claim 20, wherein the socket includes at least one of a generally hexagonally-shaped portion and a generally torqx-shaped portion.

22. The apparatus of claim 21, wherein the socket includes at least one of a screw-threaded portion and a tapered portion.

23. The apparatus of claim 8, wherein the base includes a first generally keel-like portion extending generally radially outwardly relative to the through-channel, the base includes a second generally keel-like portion extending generally radially outwardly relative to the through-channel at an angle within a range of about 210-240 degrees from the generally radially outward extension of the first generally keel-like portion, the first portion of the elongated member is retained in the through-channel at least in part by screwing the first portion of the elongated member into the base, the elongated member defines a socket, the socket includes a generally hexagonally-shaped portion, and the socket includes a screw-threaded portion.

24. The apparatus of claim 8, wherein the base includes a first generally keel-like portion arcuately extending away from the through-channel, the base includes a second generally keel-like portion arcuately extending away from the through-channel, the first portion of the elongated member is retained in the through-channel at least in part by screwing the first portion of the elongated member into the base, the elongated member defines a socket, the socket includes a generally hexagonally-shaped portion, and the socket includes a screw-threaded portion.

25. The apparatus of claim 23, further comprising:
a tibio-femoral insert coupled to the tibial plate.

26. The apparatus of claim 24, further comprising:
a tibio-femoral insert coupled to the tibial plate.

27. An implant apparatus for a proximal tibia, comprising:
a tibial plate including a superior surface, further including an inferior surface, and defining a through-hole extending from the superior surface to the inferior surface;
a first means for anchoring the apparatus in the proximal tibia, the first anchoring means being inserted into the through-hole from the superior surface such that the first anchoring means is partially retained within the through-hole and partially extends inferiorly from the tibial plate.

28. The apparatus of claim 27, further comprising:
a second means, extending inferiorly from the tibial plate, for further anchoring the apparatus in the proximal tibia.

29. The apparatus of claim 28, wherein the first anchoring means includes a first portion retained in the tibial plate, and further includes a second portion extending away from the second anchoring means.

30. For a proximal tibia with dense cancellous bone regions, an implant apparatus comprising:
a tibial plate including a superior surface, further including an inferior surface, and defining a through-hole extending from the superior surface to the inferior surface; and
a first means, extending inferiorly from the tibial plate, for anchoring the apparatus in at least one of the dense cancellous bone regions of the proximal tibia.

31. The apparatus of claim 30, further comprising:
a second means for anchoring the apparatus in the proximal tibia, the second anchoring means being inserted into the through-hole from the superior surface such that the second anchoring means is partially retained within the through-hole and partially extends inferiorly from the tibial plate.

32. The apparatus of claim 31, wherein the second anchoring means includes a first portion retained in the tibial plate, and further includes a second portion extending away from the first anchoring means.

33. A method for anchoring a first member of a tibial implant and a second member of a tibial implant in a proximal tibia, the method comprising the steps of:
anchoring the first member in the proximal tibia;
anchoring the second member in the proximal tibia; and
coupling the second member to the first member simultaneously with the step of anchoring the second member in the proximal tibia.

34. The method of claim 33, wherein the coupling step includes extending the second member through the first member.

35. A method for anchoring an implant in a proximal tibia, the implant including a first member and a second member, the first member defining a through-channel bounded by a first opening and a second opening, the second member including a post, the method comprising the steps of:
placing the first member in contact with the proximal tibia such that the first opening is positioned superior to the second opening; and
anchoring the first member to the proximal tibia;
wherein the anchoring step includes a step of inserting the post into the first opening such that the post extends inferiorly from the second opening.

36. The method of claim 35, further comprising the step of plugging the through-channel prior to the anchoring step.

37. The method of claim 35, further comprising the step of coupling the second member to the first member.

38. The method of claim 37, further comprising the step of unplugging the through-channel prior to the coupling step.

39. A method for anchoring an implant in a proximal tibia, the implant including a tibial plate and a post, the method comprising the steps of:
anchoring the tibial plate to the proximal tibia;
positioning the post superiorly to the tibial plate; and
extending the post through the tibial plate into the proximal tibia.

40. The method of claim 39, wherein the anchoring step includes a step of securing the post to the tibial plate.

41. A method for anchoring an implant in a proximal tibia, the implant including a tibial plate, the implant further including a single post having a threaded portion and a smooth portion extending from the threaded portion, the method comprising the steps of:
anchoring the tibial plate to the proximal tibia;
positioning the single post superiorly to the tibial plate;
extending the single post through the tibial plate into the proximal tibia; and
screwing the threaded portion of the single post into the tibial plate;
wherein the extending step includes anchoring the smooth portion of the single post in the proximal tibia.
